(19) United States
(12) Patent Application Publication
(10) Pub. No.: US 2009/0311666 A1
(43) Pub. Date: Dec. 17, 2009

(54) MICROFLUIDIC DEVICE FOR CRYSTALLIZATION AND CRYSTALLOGRAPHIC ANALYSIS OF MOLECULES

(75) Inventors: Claude Sauter, Strasbourg (FR); Bernard Lorber, Ittenheim (FR); Richard Giège, Strasbourg (FR); Anne Theoblad-Dietrich, Eschau (FR); Chantal Kahn-Malek, Besancon (FR); Bernard Gauthier-Manuel, Besancon (FR); Gaël Thuillier, Dijon (FR); Rosaria Ferrigno, Villeurbanne (FR)

Correspondence Address: ARENT FOX LLP, 1050 CONNECTICUT AVENUE, N.W., SUITE 400, WASHINGTON, DC, 20036, US

(73) Assignee: CENTRE NATIONAL DE LA RECHERCHE SCIENTIFIQUE-CNRS, PARIS CEDEX 16 (FR)

(21) Appl. No.: 12/374,201

(22) PCT Filed: Jul. 19, 2007
(86) PCT No.: PCT/FR2007/001243

§ 371 Date: Apr. 13, 2009

(30) Foreign Application Priority Data
Jul. 19, 2006 (FR) 0606583

(51) Int. Cl. C12Q 1/70; B01D 9/02; C07K 1/00; C12N 7/02; G01N 33/00

(52) U.S. Cl. 435/5; 422/245.1; 422/253; 530/402; 435/239; 436/86

(57) ABSTRACT

The present invention relates to a microfluidic device comprising at least one crystallization chamber adapted for comprising a solution in which at least one compound is present according to a concentration gradient, and wherein the geometry of said crystallization chamber allows for convection phenomena to be limited. The invention also relates to the use of said device, in particular for crystallization by counter diffusion and to a crystallization method.

The present invention relates to the field of crystallization. It more particularly relates to a microfluidic device for the crystallization and the crystallographic analysis of molecules, more particularly biological molecules.

The development of devices making it possible to obtain high quality crystals is an important challenge, more particular in the field of biology, most particularly in the field of structural genomics, chemistry, pharmacy and medicine, more particularly for research in the drug discovery area.

The crystallization of molecules and more particularly biological molecules is a complex multi-parametric process which involves a large number of physico-chemical and biochemical variables. Thus, the research for and the optimisation of conditions suitable for obtaining high quality crystals can require an important quantity of substances to be crystallized such as biomolecules or synthetic compounds, a material which can be very expensive.

At present, the research for devices aiming at obtaining high quality crystals with a small quantity of sample to be crystallized is in full expansion.

Thus, since 2003, the Californian Corporation Fluidigm provides a microfluidic chip making it possible to prepare high quality crystals from a small quantity of sample. However, this device must be manually filled, and is operated with a pressure-operated valve system, which makes using the device complex and which can be a source of breakdowns. In addition, this device is expensive and hardly allows an in situ analysis of the crystals.

Although some miniaturized devices are already used for the crystallization of molecules, they can be very expensive, not reliable enough, difficult to use, not adapted to both the screening and the optimisation of the crystallization conditions, or not even allow in situ analysis of the crystals in particular by X-ray diffraction.

Thus, there remains a need for devices having enhanced properties for the crystallization of molecules.

The inventors have now developed a device making it possible to wholly or partially solve the aforementioned problems.

According to a first aspect, the invention provides a microfluidic device comprising at least one crystallization chamber that may comprise a solution in which at least one compound is present according to a concentration gradient and in which the geometry of the crystallization chamber allows for convection phenomena to be limited.

Thus, the invention also relates to a crystallization chamber intended to receive a solution comprising a crystallization agent. Consequently, the invention also relates to a crystallization chamber comprising this solution or not, as well as a microfluidic device comprising at least one chamber with or without said solution.

“Microfluidic device”, according to the present invention, refers to a miniaturized device using a very small quantity of liquid sample, in the order of the microliter, or even smaller than a microliter. Inside the device, the geometry and the reduced dimensions of said crystallization chamber minimize convection movements in the solutions, such as observed, for example, in interferometry. The microfluidic environment thus favours a more homogenous crystalline growth in a medium limiting convection phenomena, or even free of convection phenomena.

“Crystallization chamber”, according to the invention, refers to a chamber adapted to the crystallization of molecules, more particularly a liquid- and gas-tight area, and most particularly a water-tight, volatile solvent-tight (such as alcohols), steam-tight and/or air-tight area.

More particularly, the crystallization chamber is connected to at least one tank (R1).

“Tank”, according to the present invention, refers to an airtight enclosure suitable for containing a fluid, the volume of which can be greater than that of the crystallization chamber.

The crystallization chamber, according to the invention, can be so arranged as to allow batch crystallization or crystallization by counter-diffusion, preferably crystallization by counter-diffusion.

The technique of the crystallization by counter-diffusion was developed by Garcia-Ruiz in 1994 (Garcia Ruiz & Moreno, 1994, Acta. Cryst. D50, 484-490), and this technique is well known to the persons skilled in the art.

More particularly, the crystallization chamber according to the invention has a section or a diameter, smaller than or equal to 400 [symbol] m, particularly smaller than or equal to 300 [symbol] m, more particularly smaller than or equal to 200 [symbol] m, or even smaller than or equal to 100 [symbol] m.

The crystallization chamber according to the invention can have a length greater than or equal to 10 mm, more particularly greater than or equal to 30 mm.

The crystallization chamber according to the invention can have a length/width ratio, greater than or equal to 10, more particularly greater than or equal to 100 and most particularly greater than or equal to 1,000.

The crystallization chamber according to the invention can have a square, rectangular, hemispherical, triangular or tubular section, more particularly a square or rectangular cross-section.

More particularly, the geometry of the crystallization chamber according to the invention includes means for improving crystallization, more particularly for increasing the number of crystals formed, more particularly through chemical function grafting, fillers, enzyme substrates and/or ligands, or through particular geometrical arrangements such as baffles, asperities or surface irregularities.

Techniques related to grafting chemical functions, fillers, enzyme substrates and/or ligands are techniques well known to the persons skilled in the art (Ulman, 1991, Introduction to thin organic films: From Langmuir-Blodgett to self Assembly; Academic Press, Boston).

Thus, the device according to the invention can more particularly enable the crystallization of macromolecules such as enzymes, nucleic acids or membrane proteins.

The crystallization chamber according to the invention can be made at least by lithography, micro-machining, injection moulding, press moulding, hot or cold press casting and/or printing methods.

“Lithography method” refers to a method derived from the semi-conductor industry, the general principle of which consists in creating an image on a substrate covered with a layer of sensitive material such as described by Chang and Sze (1996, ULSI technology, Mac Graw-Hill International Editions) and by Xia and Whitesides (1998, Annu. Rev. Mater. Sci., 28, 153-184).

As examples of lithography methods, photolithography, X-Ray lithography, UVE lithography, electronic lithography, ionic lithography and nanoprinting lithography can be cited. Such techniques can be easily identified by the persons skilled in the art thanks to their general knowledge.

Micro-machining methods involving removal of material can be based on the utilisation of a cutting tool or a laser.

The material or materials making up the crystallization chamber according to the invention and its surroundings can be “transparent,” more particularly they can let the visible spectrum, incident X-rays and/or the crystal-diffracted signal through. The material or materials can more particularly be chosen from the group comprising polydimethyl-siloxane (PDMS), polymethyl-methacrylate (PMMA), polycarbonate, cyclo-olefine copolymer (COC) and resin SU8, preferably polymethyl-methacrylate.

Thus, the device according to the invention can allow for the kinetic monitoring of crystal growth for example by videomicroscopy, of the formation of a concentration gradient, more particularly by interferometry.

In particular, at least a part of the volume defined by the crystallization chamber according to the invention comprises a gel.

“Gel” refers to a dyphasic medium composed of a three-dimensional network of a cross-linked polymer impregnated with a liquid such as a molecular solution to be crystallized. Cross-linking can be of a physical origin in the case for example of gels of agarose, cellulose and/or their derivatives, or a chemical gel such as for example a silica or acrylamide-bisacrylamide gel.

The gel according to the invention can be selected from the group comprising gels of agarose, cellulose and/or their derivatives, or silica and/or acrylamide-bisacryamide gels.

In particular, at least a part of the volume defined by one end of the crystallization chamber according to the invention comprises a gel.

In particular, the whole volume defined by the crystallization chamber according to the invention comprises a gel.

The crystallization chamber according to the invention can have on at least a part of its inner surface, means for increasing its wettability.

“Wettability” according to the invention refers to the capacity of the surface to be wetted by an aqueous solution, which results in a contact angle smaller than 90° being observed.

Thus, the invention is remarkable in that, when the crystallization chamber has an hydrophobic surface, which is the case with elastomers or plastics, the addition of a wetting agent like surfactants makes it possible to have the aqueous solutions spontaneously enter the crystallization chamber, including those containing proteins, more particularly when the crystallization chamber is in the form of channels. Thus, the addition of these surfactants makes it possible to increase the wetting power of the samples and to get rid of the valve and pump systems since the samples penetrate the crystallization chamber by capillarity.

Another solution according to the invention consists in chemically modifying the surface of the chamber so as to make it more hydrophilic.

Thus and as examples of means for increasing wettability, the following can be mentioned:
i) surface modification by physical or chemical treatments or a combination of both, for example plasma treatments, more particularly oxygen, ozone, ultra-violet treatment, ions treatments, absorption of surfactants, grafting of hydrophilic groups;
ii) the addition of surface-active molecules to an aqueous solution.

According to a particular embodiment, the crystallization chamber may be filled by capillarity.

“Capillarity” according to the invention refers to a phenomenon visible through the raising of the fluid in a tube with a small diameter.

“Solution” according to the present invention refers to an homogeneous liquid comprising at least one solvent and one solute, said solute being dissolved in the solvent.

“Compound” according to the present invention refers to a chemical substance.

More particularly, said compound is a crystallization agent.

“Crystallization agent” according to the present invention refers to an organic or inorganic, naturally-occurring or synthetic compound, favoring the crystallization of molecules.

As examples of crystallization agents, salts like sodium chloride, ammonium sulphate, alcohols such as methyl-2,4-pentanediol, ethanol, polymers such as polyethylene-glycols and their derivatives, as well as polyamines can be cited.

“Concentration gradient” according to the invention refers to a variation in the concentration of a compound from the most concentrated medium to the least concentrated medium.

More particularly, the microfluidic device makes it possible to obtain a gradient in compound concentration, and more particularly in crystallization agent concentration, ranging from a concentration lower than or equal to 25%, particularly 20%, even 15%, particularly 10%, more particularly 5%, or even 0%, to a concentration higher than or equal to 50%, or even 75&, particularly 85%, more particularly 95% and very particularly 100% of the compound saturation concentration and more particularly of the crystallization agent saturation concentration.

More particularly, said concentration gradient is established on at least 20% of the length of the crystallization chamber, more particularly on at least 40%, more particularly on at least 60%, very particularly on at least 80% or even on the whole length of the crystallization chamber.

Thus, the device according to the invention makes it possible to obtain a very wide variety, a continuum, of crystallization conditions. In particular, the device makes it possible to obtain a continuous or almost continuous variation of the conditions.

More particularly, when the device according to the invention includes several crystallization chambers, said compound, more particularly the crystallization agent, is present at a different concentration in each crystallization chamber.

Thus, when crystallization conditions are identified, this device can be used for optimizing them.

By limiting conduction phenomena, the device according to the invention, can make it possible to obtain high quality crystals with very small quantities of materials to be crystallized.

“Convection phenomena” according to the present invention, refers to the movements within a fluid resulting for example from a variation in the temperature or density.

“Geometry” according to the present invention refers to the elements' arrangement in space, more particularly the arrangement of said crystallization chamber.

More particular, the device according to the invention can enable the crystallization of molecules in a medium free of air and/or gas that lead(s) to the degradation of compounds and more particularly of molecules to be crystallized. This can also enable the crystallization of sensitive molecules, more particularly oxidation sensitive molecules.

The device according to the invention comprises at least one solution including a surface-active substance, more particularly selected from the group comprising non-ionic and zwitterionic surfactants, more particularly making it possible to solubilise the molecules to be crystallized.

“Surface-active substance” according to the present invention refers to a chemical compound having surface-active proprieties.

As examples of surface-active substances, octylglucoside, octylthioglucoside, nonylglucoside, LDAO (lauryl-diamine oxide), Triton X-100® (polyoxyethylene octyl phenyl ether), CHAPS (3((3-cholamidopropyl) dimethylammonio)-propanesulfonic acid) and their derivatives, more particularly octylglucoside, can be cited.

The concentration in surface-active substance can vary depending on the selected product, more particularly from 1 to 100% or more than the critical micellar concentration (CMC).

For example, the CMC of octyglucoside in water is 20 mM, that of octylthioglucoside is 6.5 mM, that of nonylglucoside is 9.5 mM, that of LDAO is 2 mM, that of Triton X-100® is 0.9 mM and that of CHAPS is 8 mM.

More particularly, the device according to the invention does not include:
mechanical means for filling the crystallization chamber in particular, like valves and pressure means, and/or
movable parts,
   more particularly for enabling the use of said device in particular during the filling of the crystallization chamber.

Thus, the device according to the invention can be easily used, have an enhanced reliability and/or reduced production costs.

Advantageously, the device according to the invention can enable an in situ analysis of the crystals present in the crystallization chamber by X-ray diffraction.

The device according to the invention can be transparent or translucent to light, more particularly to enable the observation of crystals with a naked eye, with an optical magnification, more particularly with an optical magnification.

More particularly, said solution according to the invention further comprises at least one molecule of interest, of chemical, biological, medical and/or pharmaceutical origin, more particularly an inorganic or organic molecule, a naturally-occurring or a synthetic macromolecule, more particularly selected from the group comprising nucleic acids, proteins, supramolecular complexes and viruses.

The microfluidic device according to the invention can include means for obtaining a given temperature in the whole device or in at least one crystallization chamber.

The microfluidic device according to the invention can include means for obtaining a temperature gradient in at least one part of at least one crystallization chamber, more particularly on the whole length of at least one crystallization chamber and most particularly in the whole device according to the invention.

Such means can be easily identified by the persons skilled in the art using their general knowledge.

As example of means for obtaining a given temperature in the whole device or in at least one crystallization chamber, the utilisation of Peltier elements can be cited. “The Peltier” effect refers to a heat displacement effect in the presence of an electric current in conducting materials of a different nature and connected by junctions. One of the junctions slightly cools down, whereas the other junction heats up.

More particularly, the microfluidic device according to the invention can include means for obtaining a temperature gradient in at least one part of at least one crystallization chamber, or even on the whole length of at least one crystallization chamber.

Such means can be easily identified by the persons skilled in the art using their general knowledge.

As examples of means making it possible to obtain a temperature gradient in at least one part of at least one crystallization chamber or even on the whole length of at least one crystallization chamber, the Peltier elements can be cited.

According to another aspect, the invention also provides the use of the device according to the invention for one of the following applications:
crystallization by counter-diffusion,
research for new active principles and/or new forms of active principles, more particularly new crystalline forms,
research by screening and optimization of crystallization conditions,
   more particularly, in the case of molecules of interest such as salts or organic or inorganic molecules, biological macromolecules, virus or drug active principles.

Thus, the device according to the invention can be used for screening and optimizing molecules crystallization conditions.

According to another aspect, a further object of the invention is the use of the device according to the invention within a system making it possible to carry out an X-ray diffraction analysis of the crystals present in the crystallization chamber.

Thus, the device according to the invention can be used for analysing crystals in situ without any handling that could affect their quality.

According to another aspect, a further object of the invention is a crystallization method comprising at least the steps consisting in:

(i) depositing at one end of a crystallization chamber a solution comprising at least one molecule of interest, more particularly a macromolecule,
(ii) depositing at an other end of the crystallization chamber a solution comprising at least one crystallization agent, then
(iii) letting crystals form.

More particularly, in the method according to the invention, said crystallization chamber is included in a device according to the invention.

More particularly, the crystallization method according to the invention further includes a step consisting in:

(iv) depositing at one end of a crystallization chamber a solution comprising a compound selected from the group comprising enzymes substrates, ligands, cryoprotectants, compounds facilitating the determination of a tridimensional structure such as heavy atoms.

Other advantages and characteristics of the invention will become apparent to the reader while referring to the Figures and to the examples that follow.

The following Figures and Examples are not limiting and are given for illustration purpose:

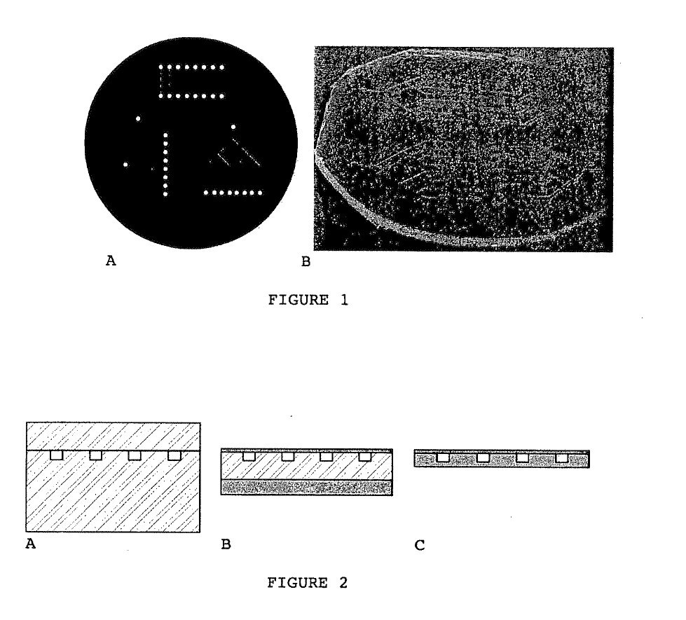

FIGS. 1 (A and B) illustrates devices according to the invention made of PDMS. FIG. 1A illustrates, in the form of a diagram, a mask having 3 types of geometries of a crystallization chamber in the shape of isolated channels, a comb and a tree. FIG. 1B shows a PDMS substrate including four cast-moulded devices with a tree-shaped geometry.

FIGS. 2 (A, B and C) illustrates, in the form of a diagram, three types of devices according to the invention. FIG. 2A illustrates a device having a total thickness of 4-5 mm, comprising a layer of PDMS (shown with hashed area) in which the crystallization chambers are moulded in the shape of channels and are closed by gluing a second layer of PDMS. FIG. 2B illustrates a device formed by a 0.5-1 mm thick layer of PDMS, in which the crystallization chambers are moulded in the shape of channels and which are closed by a transparent plastic film (shown in dark grey). The layer of PDMS is rigidified by a support-layer made of PMMA (shown in light grey). FIG. 2C illustrates a 0.25 mm thick device formed by a layer PMMA in which the crystallization chambers are moulded in the shape of channels and are closed by a transparent plastic film.

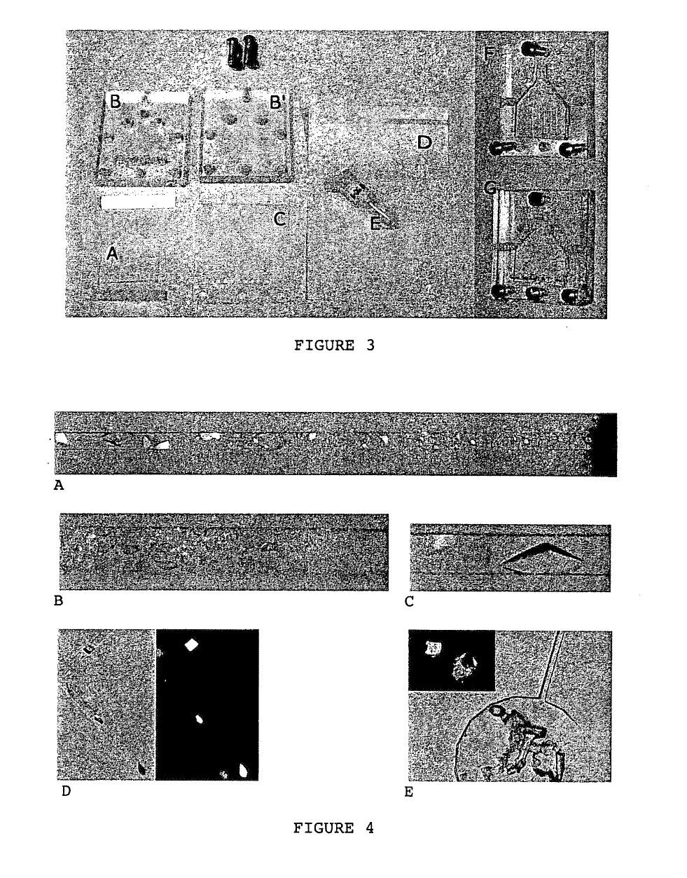

FIG. 3 illustrates the filling with PDMS of a device according to the invention.

FIGS. 4 (A, B, C, D and E) illustrates, in the form of pictures, the formation of crystals of the turnip yellow mosaic virus (TYMV), thaumatin, chicken and turkey lysozymes in devices according to the invention.

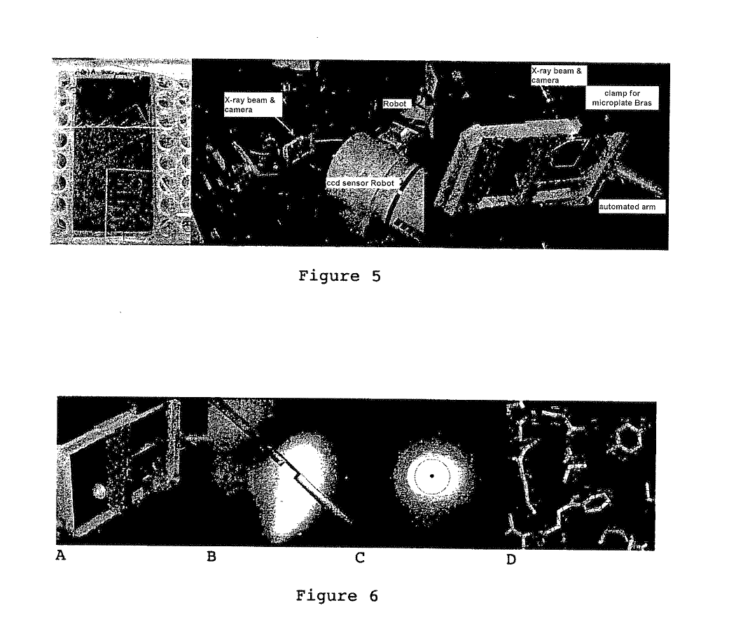

FIG. 5 illustrates the positioning of the device according to the invention on a line of synchrotron light for an X-ray analysis. The device was fixed on a standard microplate (plate NUNC with 96 wells in removable rows), the assembly being positioned in the X-ray beam, 200 mm away from the MAR CCD detector and held in position by the clamp of the handling arm of a robot (Stäubli, France).

FIG. 6 (A to D) illustrates the in situ analysis of chicken lysozyme crystals by X-rays diffraction. FIG. 6A shows a device according to the invention made of PMMA, the crystallization chambers of which are positioned in tree-like fashion. The device is fixed on a microplate and held in position by a clamp. FIG. 6B shows a chicken lysozyme crystal observed through a forward-viewing camera. FIG. 6C is a diffraction pattern, the resolution ranges of which are indicated by circles at 2.1 Å, 2.8 Å, 4.3 Å and 8.5 Å.

FIG. 6D shows an electronic density map (with a resolution of 2.15 Å) with the atomic model of the protein.

### EXAMPLES

### I. Examples 1

### Manufacturing of Devices

### I.1 Manufacturing of Devices in Polydimethyl-siloxane (PDMS)

Microfluidic devices have been manufactured in polydimethyl-siloxane (PDMS) in four successive steps:

A mask on the transparent film was obtained by laser printing.

A mould in a thick resin SU8 was then made by photolithography from said mask (FIG. 1B).

The devices were then obtained by moulding.

The crystallization chambers are then sealed by gluing a second layer of PDMS or a transparent plastic film such as Viewseal®, Clearseal®, Mylar®.

Devices containing crystallization chambers having various geometries have been manufactured as shown in FIG. 1A: crystallization chambers in the form of either isolated channels or channels having a comb- or tree-shape.

FIG. 1B shows the moulding of four devices made of PDMS, the crystallization chambers of which have a tree-shaped geometry.

I.2 Manufacturing of Devices made of polymethyl-methacrylate (PMMA)

Devices according to the invention have been manufactured from polymethyl-methacrylate (PMMA) by laser ablation (etching of crystallization chambers in a 250 μm thick layer of PMMA). The crystallization chambers were then closed by a transparent plastic film (FIG. 2C).

The devices according to the invention manufactured out of PMMA turned out to be particularly well suited for a crystallographic analysis, more particularly by X-rays diffraction, and offer numerous advantages with respect to devices made of PDMS.

### II. Filling of the Devices

### II.1 Two Main Techniques for Filling the Devices

The crystallization chambers of the device according to the invention were filled by capillarity, more particularly according to two techniques:

1) a drop of solution containing a molecule to be crystallized, a gelling agent such as agarose (0.2%-0.5% w/v and a surface-active substance such as octylglucoside (0.5% w/v) was deposited at the end of a crystallization chamber which was filled by capillarity.

A solution containing a crystallization agent was then deposited at another end of the crystallization chamber.

2) A drop of a solution containing a molecule to be crystallized and a surface-active substance such as octylglucoside (0.5% w/v) was deposited at the end of a crystallization chamber which was filled by capillarity.

A solution containing a gelling agent such as agarose (2% w/v) was then deposited at another end of said crystallization chamber.

Finally, a solution containing a crystallization agent was then deposited on the gel at the same other end of the crystallization chamber.

The surface-active substance made it possible to stabilise the molecules and more particularly the macromolecules.

The gelling agent made it possible to immobilize the solutions and the crystals in the crystallization chambers.

In addition, it made it possible to further reduce the convection phenomena and thus to favour the growth of high quality crystals.

II.2 Filling of Devices made of PDMS

The filling of a device made of PDMS is illustrated in FIG. 3.

A layer of PDMS comprising the channel-shaped crystallization chambers were deposited on a thin plate made of PMMA (C) placed on the side opposite the channels. The latter are closed by a transparent plastic film (D) such as ViewSeal®, ClearSeal® and Mylar®. This assembly was then screwed onto a 5 mm thick support made of PMMA (B2). A drop of solution containing a molecule to be crystallized, a gelling agent such as agarose (0.2%-0.5% w/v) and a detergent such as octylglucoside (0.5% w/v) was deposited at the end of the tree-shaped channels which were filled by capillarity (F).

The assembly (A) was then sandwiched between the two screwable plates made of PMMA (B and B2), and a solution containing a crystallization agent was then deposited in tanks connected to the crystallization chambers.

The assembly was then sealed by a transparent film to guarantee the tightness seal.

### III. Crystallization of YTMV Virus, thaumatine, Chicken and Turkey Lysozyme in Devices According to the Invention

Using the previously described protocols, crystals from three different proteins and one virus were obtained by counter-diffusion.

Thaumatine crystals (22 kDa) are shown in FIGS. 4A and 4C. The concentration gradient of the crystallizing agent was established by diffusion from the right to the left. The size of the crystals increases and their number decreaases as the concentration of the crystallizing agent diminishes along the channel-shaped crystallization chamber. FIG. 4C is a closer view of bipyramid-shaped thaumatine crystals obtained in 3 days in a channel-shaped crystallization chamber according to the invention, having a 100 [symbol] m channel section.

Crystals of TYMV virus, turnip yellow mosaic virus (5.106 kDa) are shown in FIG. 4B.

Quadratic crystals of chicken lysozyme obtained in a device made of PDMS including crystallization chambers in the form of isolated channels are shown in FIG. 4D. Crystals are readily visible in polarized light.

Hexagonal crystals of turkey lysozyme obtained in a device made of PDMS including crystallization chambers in the form of tree-shaped channels are shown in FIG. 4E. The crystals are readily visible when a polarizer and an analyser are crossing each other (see insert picture).

These results show that the device according to the invention enable the crystallization of proteins by counter-diffusion.

In addition, the devices according to the invention made of PDMS and PMMA are transparent enough to enable the observation of crystals with the naked eyes or using a microscope, including under polarized light.

In addition, the crystals obtained in devices according to the invention have sizes greater than 50 μm which are compatible with a direct analysis by X-rays diffraction.

### IV. Direct Analysis of Crystals by X-rays Diffraction

The crystals of chicken lysozyme have been analysed in situ by X-ray diffraction (FIG. 6). This analysis was carried out under a synchrotron X radiation at the ESRF in Grenoble.

Such crystals were obtained using the batch technique in 250 [symbol] m-size devices according to the invention and made of PMMA, and closed by a plastic film (FIG. 2C).

The devices were thus filled with 3 [symbol] l of the crystallization mixture having the following composition:
5 [symbol] l of 80 mg/ml lysozyme in 100 mM sodium acetate, pH 4.6
3 [symbol] l of agent X: 100 mM sodium acetate, pH 4.6, 1M NaCl, 30% PEG 3350)
0.3 [symbol] l of octyl-glucoside 10% (w/v) or 0.3% (w/v)
1.7 [symbol] l of buffer solution 100 mM of sodium acetate, pH 4.6.

The device was then fixed on a standard microplate (plate NUNC with 96 wells in removable rows), the resulting assembly being positioned in the X-ray beam, 200 mm away from the MAR CCD detector and being held by the handling arm of a robot (Stäubli, France) (FIG. 5).

A set of thirty successive pictures taken on one of the crystals (exposure conditions: 20 seconds, distance of 200 mm, wavelength 0.800 Å) made it possible to calculate an electronic density map with a resolution of 2.15 Å and to determine of a three-dimensional structure of the protein (FIG. 6D).

The crystallographic data can be summed-up as follows:

| Wavelength | 0.799 | Å |
|---|---|---|
| Distance | 200 | mm |
| Exposure | 15 | s |
| Oscillation | 1° | |
| Number of pictures | 30 | |
| Space group | P43212 | |
| Crystal cell parameters | a = 79, 1 Å, c = 38.8 Å | |
| Resolution | 2.15-20 | Å |
| Number of reflections (unique) | 12115 | (5040) |
| Completeness | 72.4% | (72.7%)* |
| Rsym | 7.4% | (20.1%)* |

*high resolution data: 2.15-2.21 Å

All these experiments show that the device according to the invention makes it possible to obtain high quality crystals from a small quantity of samples to be crystallized, In addition, the device according to the invention makes it possible both to screen and to optimise crystallization conditions, video-microscopy monitoring and in situ X-rays diffraction analysis of crystals. The ease operation and the geometry of the devices should facilitate the automation of all steps, more particularly with respect to high throughput applications for structural genomics.

1. A microfluidic device comprising at least one crystallization chamber adapted for comprising a solution in which at least one compound is present according to a concentration gradient, and wherein the geometry of said crystallization chamber allows for convection phenomena to be limited.

2. A microfluidic device according to claim 1, characterized in that at least one compound is present according to a concentration gradient ranging from a concentration lower than or equal to 25%, particularly 20% or even 15%, or more particularly 10%, very particularly 5% or even 0%, to a concentration higher than or equal to 50% or even 75% particularly 85%, more particularly 95% and most particularly 100% of compound saturation concentration.

3. A microfluidic device according to one of claims 1 or 2, characterized in that the crystallization chamber is connected to at least one tank (R1).

4. A microfluidic device according to claim 1, characterized in that the crystallization chamber is so arranged as to allow for crystallization by counter-diffusion.

5. A microfluidic device according to claim 1, characterized in that said compound is a crystallization agent.

6. A microfluidic device according to claim 1, characterized in that said concentration gradient is established on at least 20% of the length of the crystallization chamber, particularly on at least 40%, more particularly on at least 60% and most particularly on at least 80%, or even on the whole length of the crystallization chamber.

7. A microfluidic device according to claim 1, characterized in that the crystallization chamber has a section or a diameter smaller than or equal to 400 [symbol] m, more particularly smaller than or equal to 300 [symbol] m, most particularly smaller than or equal to 200 [symbol] m, or even smaller than or equal to 100 [symbol] m.

8. A microfluidic device according to claim 7, characterized in that the crystallization chamber has a length greater than or equal to 10 mm, more particularly greater or equal to 30 mm.

9. A microfluidic device according to claim 1, characterized in that the crystallization chamber has a length/width ratio greater than or equal to 10, more particularly greater than or equal to 100 and most particular the greater than or equal to 1,000.

10. A microfluidic device according to claim 1, characterized in that at least one part of the volume defined by the crystallization chamber comprises a gel.

11. A microfluidic device according to claim 1, characterized in that at least one part of the volume defined by one end of the crystallization chamber comprises a gel.

12. A microfluidic device according to one of claims 10 or 11, characterized in that said gel is selected from the group comprising the gels of agarose, cellulose and/or their derivatives, or silica and/or acrylamide-bisacrylamide gels.

13. A microfluidic device according to claim 1, characterized in that it includes at least a solution including a surface-active substance more particularly selected from the group comprising non-ionic and zwifterionic surface-active agents.

14. A microfluidic device according to claim 1, characterized in that the crystallization chamber is adapted to be filled by capillarity.

15. A microfluidic device according to claim 1, characterized in that it lacks:
mechanical filling means, more particularly for filling the crystallization chamber, like valves and pressure means and/or
movable parts,
more particularly to allow the use of said device, most particularly upon the filling of the crystallization chamber.

16. A microfluidic device according to claim 1, characterized in that the geometry of the crystallization chamber includes means for improving the crystallization, more particularly for increasing the number of formed crystals selected from the group consisting of chemicals function grafting, fillers, or enzyme substrates and/or ligands, particular geometrical arrangements such as asperities or surface irregularities.

17. A microfluidic device according to claim 1, characterized in that it enables in situ analysis of the crystals present in the crystallization chamber by X-ray diffraction.

18. A microfluidic device according to claim 17, characterized in that the material or materials, making up the crystallization chamber and its surroundings is or are transparent, more particularly let the visible spectrum, the incident X-rays and/or the crystal-diffracted signal through.

19. A microfluidic device according to claim 1, characterized in that the material or materials making up the crystallization chamber is or are selected from the group comprising polydimethyl-siloxane (PDMS), polymethyl-methacrylate (PMMA), polycarbonate, cyclo-olefine copolymer(COC), resin SUB, preferably polymethyl-methacrylate.

20. A microfluidic device according to claim 19, characterized in that it is transparent or translucent to light, more particularly to enable the observation of crystals with the naked eye, with an optical magnification, more particularly with an optical magnification.

21. A microfluidic device according to claim 1, characterized in that the crystallization chamber has a square, rectangular, hemispherical, triangular or tubular more particularly a square or rectangular cross-section.

22. A microfluidic device according to claim 1, characterized in that the crystallization chamber is obtained through at least a lithography, micro-machining, injection moulding, press moulding, hot or cold press casting and/or printing method.

23. A microfluidic device according to claim 1, characterized in that said solution further comprises at least one molecule of interest, of chemical, biological, medical and/or pharmaceutical origin, more particularly an inorganic or organic molecule, a naturally-occurring or synthetiv macromolecule, more particularly selected from the group comprising nucleic acids, proteins, supramolecular complexes and viruses.

24. A microfluidic device according to claim 1, characterized in that it includes means for obtaining a given temperature in the whole device or in at least a crystallization chamber.

25. A microfluidic device according to claim 1, characterized in that it includes means for obtaining a temperature gradient in at least one part of at least one crystallization chamber, more particularly on the whole length of at least one crystallization chamber and more particularly in the whole said device.

26. The use of the device such as defined according to claim 1 for any one of the following applications:
crystallization by counter-diffusion,
research for new active principles and/or new forms of active principles, more particularly new crystalline forms,
research by screening and optimisation of the crystallization conditions, more particularly in the case of molecules of interest, such as salts, organic molecules, inorganic molecules, biological macromolecules, viruses or drug active principles.

27. The device according to claim 1 together with a system enabling the analysis by X-ray diffraction of the crystals present in the crystallization chamber.

28. A crystallization method comprising at least the following steps:
(i) depositing, at one end of a crystallization chamber, a solution comprising at least one molecule of interest, more particularly a macromolecule,
(ii) depositing, at another end of the crystallization chamber, a solution comprising at least one crystallization agent, then
(iii) letting crystals form and characterized in that said crystallization chamber is included in a device comprising at least one crystallization chamber adapted for comprising a solution in which at least one compound is present according to a concentration gradient, and wherein the geometry of said crystallization chamber allows for convection phenomena to be limited.
